(19) United States
(12) Patent Application Publication
(10) Pub. No.: US 2025/0281604 A1
(43) Pub. Date: Sep. 11, 2025

(54) mRNA vaccine for porcine Deltacoronavirus, preparation method and application thereof

(71) Applicant: Jiangsu Academy of Agricultural Sciences, Nanjing (CN)

(72) Inventors: Bin LI, Nanjing (CN); Jizong LI, Nanjing (CN); Wei WANG, Nanjing (CN); Jianhao JIANG, Nanjing (CN); Baochao FAN, Nanjing (CN); Rongli GUO, Nanjing (CN); Li XIAO, Nanjing (CN); Lixiang ZHAO, Nanjing (CN); Tianyi ZHONG, Nanjing (CN)

(73) Assignee: Jiangsu Academy of Agricultural Sciences, Nanjing (CN)

(21) Appl. No.: 19/073,006

(22) Filed: Mar. 7, 2025

(30) Foreign Application Priority Data
Mar. 8, 2024 (CN) 202410266338.5

(51) Int. Cl. A61K 39/225; A61K 9/1272; A61K 9/1277; A61K 9/51; A61K 39/00; A61P 31/14; C12N 7/00

(52) U.S. Cl. CPC A61K 39/225; A61K 9/1272; A61K 9/1277; A61K 9/5123; A61K 9/5192; A61P 31/14; C12N 7/00; A61K 2039/53; A61K 2039/552; A61K 2039/55555; C12N 2770/20034

(57) ABSTRACT

An mRNA vaccine encapsulated with lipid nanoparticles for PDCoV, a preparation method and an application thereof are provided, and the mRNA vaccine for PDCoV includes at least one of sequences as described in SEQ ID NO: 1-2; the lipid nanoparticle-encapsulated mRNA vaccines includes the mRNA vaccine, and lipids for encapsulating the mRNA vaccine, and the lipids are formed by mixing ionizable liposome, distearoyl phosphatidylcholine, cholesterol and PEG liposome.

### CROSS-REFERENCE TO RELATED APPLICATIONS

This application claims priority to Chinese Patent Application No. 202410266338. 5, filed on Mar. 8, 2024, the contents of which are hereby incorporated by reference.

### INCORPORATION BY REFERENCE STATEMENT

This statement, made under Rules 77(b)(5)(ii) and any other applicable rule incorporates into the present specification of an XML file for a “Sequence Listing XML” (see Rule 831(a)), submitted via the USPTO patent electronic filing system or on one or more read-only optical discs (see Rule 1.52(e)(8)), identifying the names of each file, the date of creation of each file, and the size of each file in bytes as follows:
File name: 138993-5003-US_SequenceListing
Creation date: 2025 Feb. 11
Byte size: 9,637

### TECHNICAL FIELD

The present disclosure relates to biomedical technology, and in particular to an mRNA vaccine for porcine deltacoronavirus, a preparation method and an application thereof.

### BACKGROUND

Porcine deltacoronavirus (PDCoV) is a novel porcine enteropathogenic coronavirus belonging to the newly discovered genus deltacoronavirus of the Coronaviridae, and causes diarrhea, vomiting, dehydration, and even death in pigs. The full-length genome of PDCOV is approximately 25.4 kb, encoding 15 mature nonstructural proteins (nsp2-16), four structural proteins (S, E, M, N) and three auxiliary proteins (NS6, NS7, NS7a), where the S protein has been identified as the most immunogenic antigen of PDCoV and plays an important role in the induction of neutralizing antibodies and antiviral T cell responses.

PDCOV may cause diarrhea and vomiting in lactating piglets from 5-15 days of age. The autopsy changes after PDCoV infection are similar to those of porcine epidemic diarrhea virus (PEDV) and transmissible gastroenteritis virus (TGEV) are similar. Vaccination remains the most effective method of preventing and controlling infectious diseases. Currently, there is no commercial vaccine for PDCOV, and further development of a safe and effective vaccine is important for the prevention and control of PDCoV. If the PDCoV genome continues to be mutated and mixed with other viruses, an effective vaccine has to be developed within a very short development cycle. In contrast, mRNA vaccines are made by introducing mRNAs containing encoded antigenic proteins into the human body, directly translating them to form corresponding antigenic proteins, and induces the body to generate specific immune responses and achieves preventive immunity. mRNA vaccines utilize the gene sequences of viruses rather than the viruses themselves, and thus mRNA vaccines are characterized by the absence of viral components and the no risk of infection. At the same time, mRNA vaccines have a short development cycle, and novel candidate vaccine may be rapidly developed to cope with viral mutations. Thus, mRNA vaccines contribute to the development of vaccines against emerging infectious disease pandemics in a short period of time.

### SUMMARY

An objective of the present disclosure is to provide an mRNA vaccine for PDCOV, a preparation method and an application thereof, so as to solve the above-mentioned problems, and the mRNA vaccine has a short development time and high safety, and may provide immune protection for PDCOV and provide technical support for prevention and control of PDCoV.

To achieve the above objective, the present disclosure provides the following schemes.

The present disclosure provides an mRNA vaccine for PDCOV, including at least one of sequences as described in SEQ ID NO: 1-2.

The present disclosure provides a preparation method of the mRNA vaccine, including following steps:
DNA sequences for synthesizing the mRNA vaccine; and
constructing the DNA sequences onto a vector, followed by enzyme digestion, transcription, and purification to obtain the mRNA vaccine.

The present disclosure provides an mRNA vaccine encapsulated with lipid nanoparticles for PDCOV, wherein the mRNA vaccine encapsulated with lipid nanoparticles comprises an mRNA vaccine, and lipids for encapsulating the mRNA vaccine, wherein the lipids are formed by mixing ionizable liposome, distearoyl phosphatidylcholine, cholesterol and PEG liposome.

Optionally, a molar ratio of the ionizable liposome, the distearoyl phosphatidylcholine, the cholesterol and the PEG liposome is 55:(5-15):(30-40):(1-2).

The present disclosure provides a preparation method of the mRNA vaccine encapsulated with lipid nanoparticles, including following steps:
dissolving the ionizable liposome, the distearoyl phosphatidylcholine, the cholesterol and the PEG liposome in ethanol to prepare lipid ethanol solution;
diluting the mRNA vaccine with citrate buffer to prepare an mRNA aqueous solution; and
mixing the lipid ethanol solution with the mRNA aqueous solution to prepare the mRNA vaccine encapsulated with the lipid nanoparticles.

Optionally, a volume ratio of the lipid ethanol solution to the mRNA aqueous solution is 1:(2-5).

The present disclosure also provides the application of the mRNA vaccine or the lipid nanoparticle-encapsulated mRNA vaccine in the preparation of a vaccine for the prevention and control of PDCoV infection disease.

Compared with the prior art, the present disclosure has the following effects.

The RNA vaccine obtained by gene synthesis, in vitro transcription, and encapsulation of lipid nanoparticles may rapidly respond to the current epidemic of PDCOV and reduce the research and development and production cycle of new vaccine development. The RNA vaccine prepared by the present disclosure has high production efficiency and may produce high levels of neutralizing and binding antibodies to PDCOV in vivo, which may effectively reduce the occurrence of diarrhea, dehydration and vomiting in piglets, and is of great value for prevention and control of porcine diarrhea, and provides an important means for the prevention and control of PDCoV.

### BRIEF DESCRIPTION OF THE DRAWINGS

In order to more clearly illustrate the technical solutions in the embodiments of the present disclosure or the prior art, the drawings to be used in the embodiments will be briefly introduced below, and it will be obvious that the drawings in the following description are only some of the embodiments of the present disclosure, and that for a person of ordinary skill in the field, other drawings may be obtained based on these drawings without putting forth creative labor.

FIG. 1 shows expression results of mRNA vaccine in 293T cells of Embodiment 2 of the present disclosure.

FIG. 2 shows the Western blotting electropherogram of Embodiment 2 of the present disclosure, where M: standard DNA molecule, SI: mRNA-S1, S2: mRNA-S2 and C: negative control.

FIG. 3 shows the detection results of IgG antibody level in mouse serum of Embodiment 3 of the present disclosure, where PBS denotes PBS solution, mRNA-S1 denotes mRNA-S1 vaccine, and mRNA-S2 denotes mRNA-S2 vaccine.

FIG. 4 shows the detection results of neutralizing antibody levels in mice of Embodiment 3 of the present disclosure, where PBS denotes PBS solution, mRNA-S1 denotes mRNA-S1 vaccine, and mRNA-S2 denotes mRNA-S2 vaccine.

FIG. 5 shows the detection results of IFN-γ level in lymphocytes of mice of Embodiment 3 of the present disclosure, where PBS denotes PBS solution, mRNA-SI denotes mRNA-S1 vaccine, and mRNA-S2 denotes mRNA-S2 vaccine.

FIG. 6 shows the detection results of IL-4 level in lymphocytes of mice of Embodiment 3 of the present disclosure, where PBS denotes PBS solution, mRNA-S1 denotes mRNA-S1 vaccine, and mRNA-S2 denotes mRNA-S2 vaccine.

FIG. 7A shows flow cytometry analyses of the frequencies of CD3⁺CD4⁺.

FIG. 7B shows flow cytometry analyses of the frequencies of CD3⁺CD8⁺.

FIG. 7C shows flow cytometry analyses of the frequencies of B cells.

FIG. 7D shows flow cytometry analyses of the frequencies of NK cells.

FIG. 8 shows the results of the lymphocyte proliferation test for the detection of lymphocytes of the present disclosure, where PBS denotes PBS solution, mRNA-S1 denotes mRNA-S1 vaccine, and mRNA-S2 denotes mRNA-S2 vaccine.

FIG. 9A shows expression results of cytokine IFN-γ in mouse cells induced by the mRNA vaccine in Embodiment 3 in the present disclosure.

FIG. 9B shows expression results of cytokine IL-4 in mouse cells induced by the mRNA vaccine in Embodiment 3 in the present disclosure.

FIG. 10 shows the results of testing IgG antibody levels in sows of Embodiment 4 of the present disclosure, where PBS denotes PBS solution, mRNA-SI denotes mRNA-S1 vaccine, and Inactivated PDCoV+GEL01 denotes PDCOV inactivated vaccine.

FIG. 11 shows the results of testing IgA antibody levels in sows of Embodiment 4 of the present disclosure, where PBS denotes PBS solution, mRNA-SI denotes mRNA-S1 vaccine, and Inactivated PDCoV+GEL01 denotes PDCoV inactivated vaccine.

FIG. 12 shows the neutralizing antibody level in sows of Embodiment 4 of the present disclosure, where PBS denotes PBS solution, mRNA-SI denotes mRNA-S1 vaccine, and Inactivated PDCoV+GEL01 denotes PDCoV inactivated vaccine.

FIG. 13 shows the neutralizing antibody level in piglets of Embodiment 5 of the present disclosure, where PBS denotes PBS solution, mRNA-SI denotes mRNA-SI vaccine, and Inactivated PDCoV+GEL01 denotes PDCoV inactivated vaccine.

FIG. 14 shows the results of testing IgG antibody level in sera of the piglets of Embodiment 5 of the present disclosure, where PBS denotes PBS solution, mRNA-SI denotes mRNA-S1 vaccine, and Inactivated PDCoV+GEL01 denotes PDCoV inactivated vaccine.

### DETAILED DESCRIPTION OF THE EMBODIMENTS

Various exemplary embodiments of the present disclosure are now described in detail, which should not be considered a limitation of the present disclosure, but rather should be understood as a more detailed description of certain aspects, features, and embodiments of the present disclosure.

It should be understood that the terms described in the present disclosure are only intended to describe particular embodiments and are not intended to limit the present disclosure. Further, with respect to the range of values in the present disclosure, it should be understood that each intermediate value between the upper and lower limits of the range is also specifically disclosed. Each smaller range between any stated value or intermediate value within the stated range, and any other stated value or intermediate value within the stated range, is also included in this disclosure. The upper and lower limits of these smaller ranges may be independently included or excluded from the scope.

Unless otherwise indicated, all technical and scientific terms used herein have the same meaning as is commonly understood by those of ordinary skill in the art described in this disclosure. While this disclosure describes only preferred methods and materials, any methods and materials similar or equivalent to those described herein may also be used in the implementation or testing of this disclosure. All literature referred to in this specification is incorporated by reference to disclose and describe the methods and/or materials associated with said literature. In the event of a conflict with any incorporated literature, the contents of this specification shall prevail.

Various improvements and variations may be made to specific embodiments of the specification of the present disclosure without departing from the scope or spirit of the present disclosure, as will be apparent to those skilled in the art. Other embodiments obtained from the specification of the present disclosure will be apparent to those skilled in the art. The specification and embodiments of the present disclosure are exemplary only.

As used herein, the terms “comprising,” “including,” “having,” “containing” and the like are all open-ended terms, i.e., meant to include but not be limited to.

### Embodiment 1 Preparation of an RNA Vaccine for PDCoV

The S glycoprotein of PDCOV CZ2020/12 (GenBank: OK546242.1) is used as a reference amino acid sequence for mRNA vaccine design. DNA sequences shown in SEQ ID NO: 1-2 are synthesized using genes, and then mRNAs are synthesized by in vitro transcription using T7 RNA polymerase and linearized plasmid DNA templates with the sequences shown in SEQ ID NO: 1-2, and the mRNAs are named S1 and S2, respectively (this part of the experiment is synthesized by Suzhou Huiliao Biomedical Technology Co. Ltd.)

The ionizable liposome Dlin-MC3-DMA, distearoyl phosphatidylcholine (DSPC), cholesterol, and DMG-PEG2000 liposome are dissolved in ethanol at a molar ratio of 55:10:38.5:1.2 (nitrogen-phosphorus ratio of 5.67), and SI and S2 are diluted in 50 mM citrate buffer (pH=4). The lipid ethanol solution and the mRNA aqueous solution are mixed at a volume ratio of 1:3 by microfluidic equipment to prepare LNPs, named mRNA-S1 and mRNA-S2. Ethanol is removed from the sample solution by ultrafiltration, and the lipid nanoparticles (LNPs) are filtered through a 0.22 μm sterile filter, and the prepared lipid nanoparticles are stored in a refrigerator at 4° C. for spare use. In the subsequent experiments, the Dulbecco's Phosphate-Buffered Saline (DPBS) buffer is adjusted to a suitable concentration for use.

Sequence of S1 (SEQ ID NO: 1):
ATGCAGAGAGCTCTATTGATTATGACCTTACTTTGTCTCGTTCGAGCAAAGTTT
GCTGATGATCTACTCGATTTGCTCAC
CTTCCCGGGTGCACATCGCTTCTTACATAAACTCACGAGTAATTCCAGCAGTCT
CTACTCGCGGGCTAATAACTTTGATG
TTGGCGTTCTTCCTGGCTACGCCACTAAGAACGTTAACCTCTTCTCACCACTTA
CTAACTCTACTTTGCCAATTAATGGC
CTTCATCGGAGTTATCAACCACTCATGCTGAATTGTTTTACTAAAATAACTAAC
CACACTCTCAGCATGTATCTCCTACC
TAGTGATGTACAAACTTATAGCTGCGGCGGTGCCATGGTTAAATACCAGACAC
ATGATGCAGTTCGTATCATTTTAGACC
TCACTGCCACTGACCACATCTCTGTTGAAGTCGTTGGCCAGCATGGTGAAAATT
ATGTGTTTGTTTGTAGTGAGCAGTTT
ACCTACACCACTGCACTACACAAATCTACTTTCTTCTCACTTAATTCTGAGCTTT
ATTGCTTTACTAATAACACCTACTT
AGGTATTCTTCCACCTGATTTAACTGACTTTACGGTCTACCGTACTGGTCAGTT
CTATGCTAATGGTTACCTTTTAGGTA
CTTTACCTATTACGGTTAACTATGTTAGGTTGTATCGGGGTCAATTGGCTGCCA
ATAGTGCCCACTTTGCCCTAGCAAAC
CTAACCGATACACTTATAACACTTACCAATACCACTATATCGCAAATCACCTAT
TGTGATAAGTCAGTAGTTGATTCAAT
AGCATGCCAGCGCTCTTCTCACGAAGTGGAGGATGGGTTTTACTCTGACCCTA
AATCTGCCGTTAGAGCTAGGCAACGTACTATTGTTACACTACCTAAGCTCCCTG
AGCTTGAAGTAGTGCAGTTAAATATTTCTGCACACATGGATTTTGGCGAAGCC
AGACTTGACAGTGTTACCATTAATGGTAACACATCCTATTGTGTCACTAAGCCT
TACTTCAGGCTTGAAACTAACTTTAT
GTGTACAGGTTGCACTATGAATCTGCGCACTGATACCTGTAGTTTTGACCTGTC
AGCAGTAAACAATGGCATGTCATTCT
CTCAATTCTGTCTAAGCACTGAATCTGGTGCTTGTGAGATGAAAATTATTGTTA
CCTATGTATGGAATTACTTGCTAAGG
CAGCGTTTGTATGTTACTGCTGTAGAAGGCCAGACTCACACTGGAACCACTTC
AGTACATGCAACAGACACTTCTAGTGTAATCACTGATGTCTGCACTGACTACA
CTATCTATGGAGTCTCTGGTACTGGCATTATTAAGCCATCAGATCTCTTATTGC
ACAATGGCATAGCATTCACCTCTCCAACAGGTGAGCTCTATGCATTTAAAAAT
ATAACCACTGGCAAAACCCTCCAGGTC
TTACCGTGTGAAACCCCTTCTCAACTGATTGTGATAAACAACACCGTTGTCGGT
GCTATCACATCCAGTAACTCAACTGA
AAATAATAGGTTTACTACTACTATTGTCACACCTACTTTCTTTTATTCCACAAAT
GCCACCACCTTTAACTGCACCAAGC
CTGTTTTGTCCTATGGACCCATCAGCGTGTGTAGTGATGGTGCAATTGTGGGAA
CATCTACATTACAGAATACTCGACCA
TCCATAGTTTCACTATACGATGGCGAAATTGAAATACCATCTGCATTTTCCCTT
TCTGTTCAGACAGAGTATTTGCAAGT
TCAAGCAGAGCAAGTTATAGTTGATTGTTCTCAGTATGTATGCAATGGCAACA
GCCGTTGTCTACAATTACTGGCACAAT
ACACCTCAGCTTGCTCTAACATTGAAGCAGCTCTGCATTCCTCTGCACAGTTGG
ATAGCAGAGAGATTATAAATATGTTT
CAAACATCAACACAGTCCTTGCAGTTGGCTAATATTACCAACTTCAAGGGTGA
CTACAATTTTAGCAGCATAATAACCCC
CAGAATTGGTGGCAGATCTGCTATTGAAGACCTTCTTTTTAATAAAGTIGTTAC
TAGTGGCCTTGGCACTGTTGATCAGG
ACTACAAAGCCTGCTCTAGAGACATGGCCATCGCTGACTTAGTTTGTTCCCAGT
ATTACAATGGCATCATGGTTCTACCT
GGTGTTGTTGATGCTGAGAAAATGGCAATGTATACTGGCTCTCTTACTGGAGCT
ATGGTATTTGGGGGACTGACTGCTGC
AGCGGCAATACCATTCGCCACGGCAGTACAAGCCCGTCTCAATTATGTCGCAC
TGCAAACAAATGTACTACAAGAAAACCAGAAAATTCTTGCAGAATCATTTAAC
CAAGCAGTTGGCAATATATCACTTGCACTATCTTCTGTTAATGATGCCATCCAG
CAAACTTCTGAGGCTCTTAGCACCGTAGCTATTGCTATTAAAAAGATTCAAAC
AGTTGTTAATCAGCAGGGTGAGGCATT
ATCACACCTGACTGCACAGCTGTCAAACAATTTCCAAGCAATTTCGACTTCTAT
TCAAGACATTTATAACCGTCTTGAGG
AAGTAGAGGCTAACCAGCAAGTTGACCGTCTCATCACAGGACGGTTGGCTGCA
CTTAATGCATATGTTACTCAGTTACTC
AATCAGATGTCTCAGATTAGACAATCTCGATTGTTAGCTCAGCAAAAGATTAA
TGAGTGTGTCAAATCTCAGTCGTCCAG
ATACGGTTTCTGTGGAAATGGCACACATATCTTCTCACTTACACAGACTGCACC
AAATGGCATATTTTTCATGCATGCAG
TACTTGTACCCAACAAATTCACACGTGTCAACGCTTCTGCGGGTATTTGTGTGG
ATAATACGAAAGGCTACTCATTGCAG
CCTCAACTTATACTCTACCAGTTTAATAACTCCTGGAGAGTTACACCTAGAAAT
ATGTATGAACCCAGATTGCCCCGGCA
AGCTGATTTCATACAATTAACTGATTGCAGCGTTAATTTTTACAATACCACCGC
TGCTAATCTTCCCAATATTATCCCTG
ACGTTATAGATGTCAATCAAACAGTCAGTGATATTATTGACAATTTACCTACAG
CAACACCTCCTCAGTGGGATGTTGGT
ATCTATAACAACACTATTCTCAACCTCACCGTTGAGATTAATGATCTACAAGAG
CGGTCTAAAAATCTCTCACAGATTGC
AGATCGTTTACAAAATTATATTGACAATCTTAACAATACTCTAGTTGACCTTGA
ATGGCTCAACAAAGTGGAGACTTACC
TTAAATGGCCGTGGTATGTATGGCTTGCCATAGCCCTGGCTCTTATTGCATTTG
TGACAATCCTCATAACAATCTTTCTC
TGTACTGGTTGTTGTGGTGGTTGTTTTGGTTGTTGTGGCGGTTGTTTTGGCCTTT
TCTCTAAGAAGAAAAGGTATACCGA
CGACCAACCAACACCGTCCTTTAAGTTTAAGGAATGGTAG
Sequence of S2 (SEQ ID NO: 2):
ATGAAATTCTTAGTCAACGTTGCCCTTGTTTTTATGGTCGTGTACATTTCTTACA
TCTATGCGGCTGATGATCTACTCGA
TTTGCTCACCTTCCCGGGTGCACATCGCTTCTTACATAAACTCACGAGTAATTC
CAGCAGTCTCTACTCGCGGGCTAATA
ACTTTGATGTTGGCGTTCTTCCTGGCTACGCCACTAAGAACGTTAACCTCTTCT
CACCACTTACTAACTCTACTTTGCCA
ATTAATGGCCTTCATCGGAGTTATCAACCACTCATGCTGAATTGTTTTACTAAA
ATAACTAACCACACTCTCAGCATGTA
TCTCCTACCTAGTGATGTACAAACTTATAGCTGCGGCGGTGCCATGGTTAAATA
CCAGACACATGATGCAGTTCGTATCA
TTTTAGACCTCACTGCCACTGACCACATCTCTGTTGAAGTCGTTGGCCAGCATG
GTGAAAATTATGTGTTTGTTTGTAGT
GAGCAGTTTACCTACACCACTGCACTACACAAATCTACTTTCTTCTCACTTAAT
TCTGAGCTTTATTGCTTTACTAATAA
CACCTACTTAGGTATTCTTCCACCTGATTTAACTGACTTTACGGTCTACCGTACT
GGTCAGTTCTATGCTAATGGTTACC
TTTTAGGTACTTTACCTATTACGGTTAACTATGTTAGGTTGTATCGGGGTCAAT
TGGCTGCCAATAGTGCCCACTTTGCC
CTAGCAAACCTAACCGATACACTTATAACACTTACCAATACCACTATATCGCA
AATCACCTATTGTGATAAGTCAGTAGT
TGATTCAATAGCATGCCAGCGCTCTTCTCACGAAGTGGAGGATGGGTTTTACTC
TGACCCTAAATCTGCCGTTAGAGCTA
GGCAACGTACTATTGTTACACTACCTAAGCTCCCTGAGCTTGAAGTAGTGCAGT
TAAATATTTCTGCACACATGGATTTT
GGCGAAGCCAGACTTGACAGTGTTACCATTAATGGTAACACATCCTATTGTGT
CACTAAGCCTTACTTCAGGCTTGAAAC
TAACTTTATGTGTACAGGTTGCACTATGAATCTGCGCACTGATACCTGTAGTTT
TGACCTGTCAGCAGTAAACAATGGCA
TGTCATTCTCTCAATTCTGTCTAAGCACTGAATCTGGTGCTTGTGAGATGAAAA
TTATTGTTACCTATGTATGGAATTAC
TTGCTAAGGCAGCGTTTGTATGTTACTGCTGTAGAAGGCCAGACTCACACTGG
AACCACTTCAGTACATGCAACAGACACTTCTAGTGTAATCACTGATGTCTGCAC
TGACTACACTATCTATGGAGTCTCTGGTACTGGCATTATTAAGCCATCAGATC
TCTTATTGCACAATGGCATAGCATTCACCTCTCCAACAGGTGAGCTCTATGCAT
TTAAAAATATAACCACTGGCAAAACC
CTCCAGGTCTTACCGTGTGAAACCCCTTCTCAACTGATTGTGATAAACAACACC
GTTGTCGGTGCTATCACATCCAGTAA
CTCAACTGAAAATAATAGGTTTACTACTACTATTGTCACACCTACTTTCTTTTAT
TCCACAAATGCCACCACCTTTAACT
GCACCAAGCCTGTTTTGTCCTATGGACCCATCAGCGTGTGTAGTGATGGTGCAA
TTGTGGGAACATCTACATTACAGAAT
ACTCGACCATCCATAGTTTCACTATACGATGGCGAAATTGAAATACCATCTGC
ATTTTCCCTTTCTGTTCAGACAGAGTA
TTTGCAAGTTCAAGCAGAGCAAGTTATAGTTGATTGTTCTCAGTATGTATGCAA
TGGCAACAGCCGTTGTCTACAATTAC
TGGCACAATACACCTCAGCTTGCTCTAACATTGAAGCAGCTCTGCATTCCTCTG
CACAGTTGGATAGCAGAGAGATTATA
AATATGTTTCAAACATCAACACAGTCCTTGCAGTTGGCTAATATTACCAACTTC
AAGGGTGACTACAATTTTAGCAGCAT
AATAACCCCCAGAATTGGTGGCAGATCTGCTATTGAAGACCTTCTTTTTAATAA
AGTTGTTACTAGTGGCCTTGGCACTG
TTGATCAGGACTACAAAGCCTGCTCTAGAGACATGGCCATCGCTGACTTAGTTT
GTTCCCAGTATTACAATGGCATCATG
GTTCTACCTGGTGTTGTTGATGCTGAGAAAATGGCAATGTATACTGGCTCTCTT
ACTGGAGCTATGGTATTTGGGGGACT
GACTGCTGCAGCGGCAATACCATTCGCCACGGCAGTACAAGCCCGTCTCAATT
ATGTCGCACTGCAAACAAATGTACTACAAGAAAACCAGAAAATTCTTGCAGAA
TCATTTAACCAAGCAGTTGGCAATATATCACTTGCACTATCTTCTGTTAATGAT
GCCATCCAGCAAACTTCTGAGGCTCTTAGCACCGTAGCTATTGCTATTAAAAA
GATTCAAACAGTTGTTAATCAGCAGGGTGAGGCATTATCACACCTGACTGCAC
AGCTGTCAAACAATTTCCAAGCAATTTCGACTTCTATTCAAGACATTTATAACC
GTCTTGAGGAAGTAGAGGCTAACCAGCAAGTTGACCGTCTCATCACAGGACGG
TTGGCTGCACTTAATGCATATGTTACTCAGTTACTCAATCAGATGTCTCAGATT
AGACAATCTCGATTGTTAGCTCAGCAAAAGATTAATGAGTGTGTCAAATCTCA
GTCGTCCAGATACGGTTTCTGTGGAAATGGCACACATATCTTCTCACTTACACA
GACTGCACCAAATGGCATATTTTTCATGCATGCAGTACTTGTACCCAACAAATT
CACACGTGTCAACGCTTCTGCGGGTATTTGTGTGGATAATACGAAAGGCTACT
CATTGCAGCCTCAACTTATACTCTACCAGTTTAATAACTCCTGGAGAGTTACAC
CTAGAAATATGTATGAACCCAGATTGCCCCGGCAAGCTGATTTCATACAATTA
ACTGATTGCAGCGTTAATTTTTACAATACCACCGCTGCTAATCTTCCCAATA
TTATCCCTGACGTTATAGATGTCAATCAAACAGTCAGTGATATTATTGACAATT
TACCTACAGCAACACCTCCTCAGTGG
GATGTTGGTATCTATAACAACACTATTCTCAACCTCACCGTTGAGATTAATGAT
CTACAAGAGCGGTCTAAAAATCTCTC
ACAGATTGCAGATCGTTTACAAAATTATATTGACAATCATCATCATCATCATCA
CAGAATGAAGCAAATTGAAGATAAGA
TTGAAGAAATTGAATCCAAGCAAAAGAAGATTGAAAATGAAATTGCTAGAATT
AAGAAGTAG

### Embodiment 2 mRNA Expression and Preparation Characterization

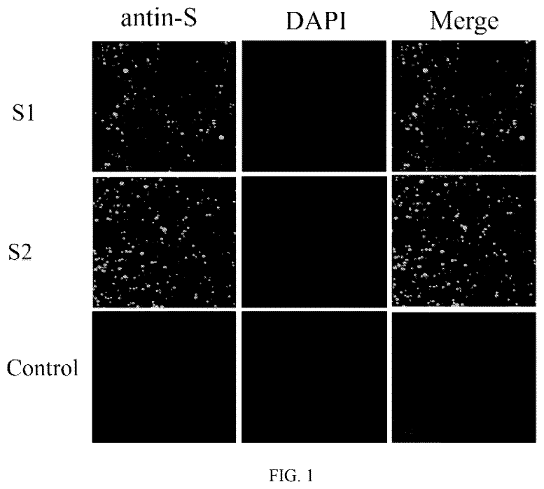

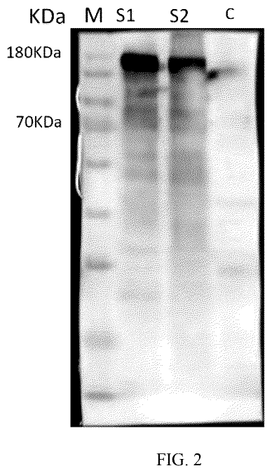

The expression of LNPs delivery mRNA is detected, and HEK293T cells are inoculated into 24-well plates. After 18 h, the cells are treated with LNPs encapsulating mRNA, where 1 ug LNPs per well, and then are incubated for 24 h. The untreated cells are used as the control group. The expression of mRNA is detected by using indirect immunofluorescence (IFA) and Western blotting, and indirect immunofluorescence detection is performed by anti-PDCoV-S polyclonal antibody, where the anti-PDCoV-S polyclonal antibody is prepared in laboratory and diluted at 1:1000, and the polyclonal antibody has been published in the literature: Zhang Baotai et al. Expression of structural protein of porcine deltacoronavirus and preparation of its polyclonal antibody). The results of IFA are shown in FIG. 1, and as may be seen from the figure, the mRNA vaccine may be efficiently expressed in 293T cells. The results of Western blotting are shown in FIG. 2, and show that the related protein may be effectively expressed.

The particle size (Size), polydispersity index (PDI) and electrodynamic potential (Zeta P) of the particles are determined by dynamic light scattering using the Malvern Nanoparticle Size Test Instrument. The encapsulation efficiency of lipid nanoparticles is determined using Quant-it Ribogreen RNA Quantification Kit (Thermo Fisher Scientific, UK). The results are shown in Table 1: most of the mRNA-S1 and mRNA-S2 lipid nanoparticle sizes are around 80 nm, most of the lipid nanoparticle potentials are near 0 mv, the polydispersity index varied around 0.1, and most of the encapsulation rates are above 95%.

TABLE 1
Characterization of lipid nanoparticles

| | | | Encapsulation |
|---|---|---|---|
| Number | Size | PDI | efficiency % |
| mRNA-S1 | 80.38 | 0.075 | 96.42 |
| mRNA-S2 | 88.72 | 0.092 | 95.33 |

### Embodiment 3 Lipid Nanoparticles-Encapsulated RNA Vaccine Immunized Mice and Antibody Level Detection

In the immunization experiments for mice, 15 6-week-old BALB/c mice are randomly divided into 3 groups, with 5 mice in each group. Each of the mice in both groups are injected subcutaneously with the mRNA vaccine at a dose of 30 ug. Mice in control group are injected subcutaneously with PBS. Vaccinated mice receive a booster dose 14 days after vaccination. All mice are necropsied on day 28. Serum is collected on days 0, 14, and 28, respectively, and antibodies in serum are detected by indirect ELISA and neutralization. Isolated splenocytes are used for lymphocyte subset ratio test, lymphopoiesis test and cytokine test by flow cytometry.

Indirect ELISA method comprises the following steps:
S1: encapsulating the purified S protein with carbonate buffer solution at pH 9.6 at a concentration of 4 ng/μL, and then adding the encapsulated purified S protein to a polystyrene microtitre plate at 100 μL/well, overnight at 4° C.;
S2: discarding the liquid the next day, adding 200 μL PBST to each well, and then washing three times for 5 min each time;
S3: preparing 5% skimmed milk with PBS, 200 μL/well, and sealing it at 37° C. for 2 h; discarding the confining liquid, and washing three times for 5 min each time by using PBST;
S4: taking serum collected after three immunizations, diluting the serum with PBS according to the ratio of 1:50, 100 μL/well, where negative serum is serum collected from mice with PBS immunization, incubating at 37° C. for 1 h, discarding the primary antibody and then washing with PBST three times, each time for 5 min;
S5: diluting Goat anti Mouse HRP-IgG with PBS at a ratio of 1:5000, 100 μL/well, incubating at 37° C. for 1 h; discarding the secondary antibody and then washing three times with PBST for 5 min each time;
S6: adding 100 μL of TMB in each well and incubating for 10 min at room temperature with light protection;
S7: adding 50 μL of the stop buffer (2 M H₂SO₄) in each well to terminate the reaction, and reading the OD_{450 nm} value with an enzyme meter;

Neutralization antibody method comprises the following steps:
S1: dividing all sera and placing it in a water bath at 56° C. and inactivating for 30 min;
S2: diluting the serum that has been inactivated on a 96-well cell culture plate at multiple proportions of 2-1, 2-2, 2-3, 2-4, 2-5, 2-6, 2-7, 2-8, respectively, with a final volume of 50 μL in each well, and do 2 replicate wells for each dilution;
S3: diluting PDCoV virus solution into 100 TCID₅₀/50 μL with dulbecco's modified eagle medium (DMEM) medium;
S4: taking 50 μL of virus solution and adding it into the above serum dilution plate, and neutralizing it for 1 h in a 5% CO₂ incubator at 37° C.;
S5: discarding the cell culture medium when the 96-well plate is full of monolayer LLC-PK 1 cells, washing with DMEM twice, adding 100 μL of mixture of serum and virus to each well, and incubating the adsorbent for 2.5 h at 37° C. in a 5% CO₂ temperature chamber; and
S6: washing with DMEM twice, replacing the pure DMEM maintenance solution containing 7.5 μg/mL trypsin, and incubating in a warm box, and starting the day-by-day observation and recording after 12 h.

At the same time, negative serum and positive serum are set as control group, and the maximum dilution of the serum that completely inhibits the cytopathic effect (CPE) is taken as the neutralizing titer of the serum.

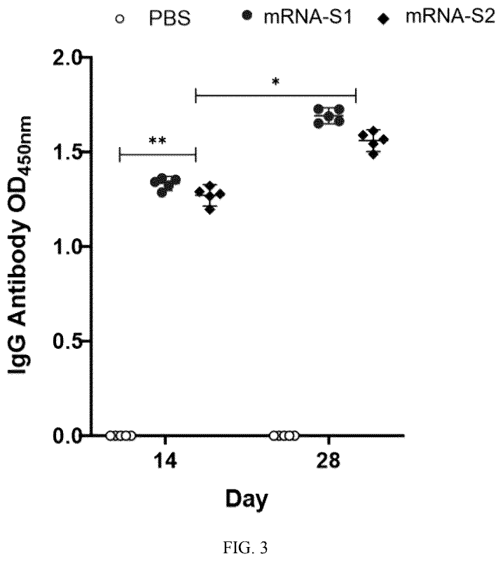

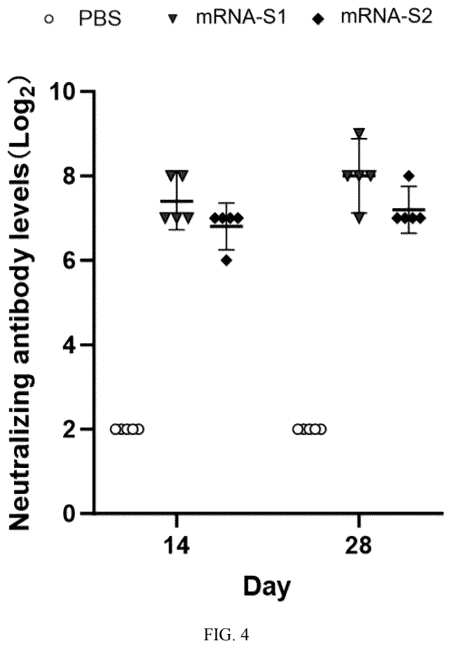

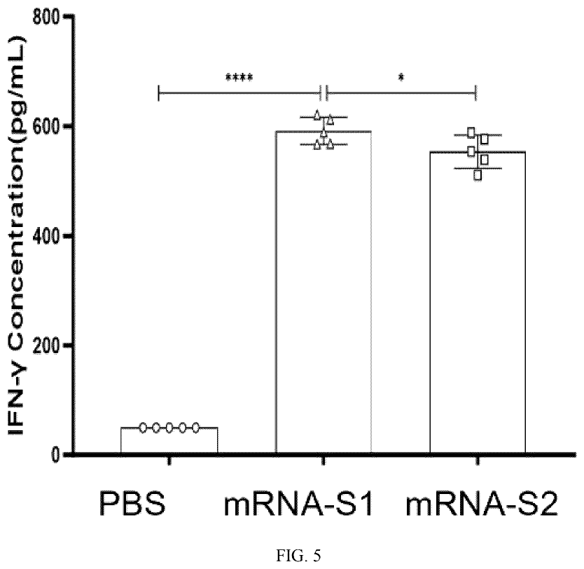

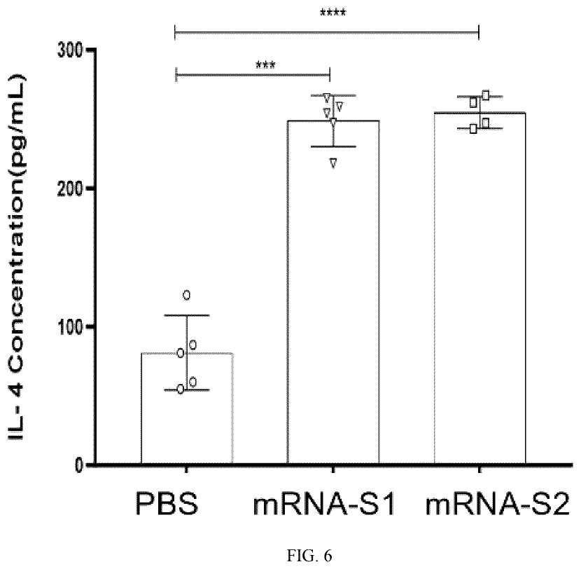

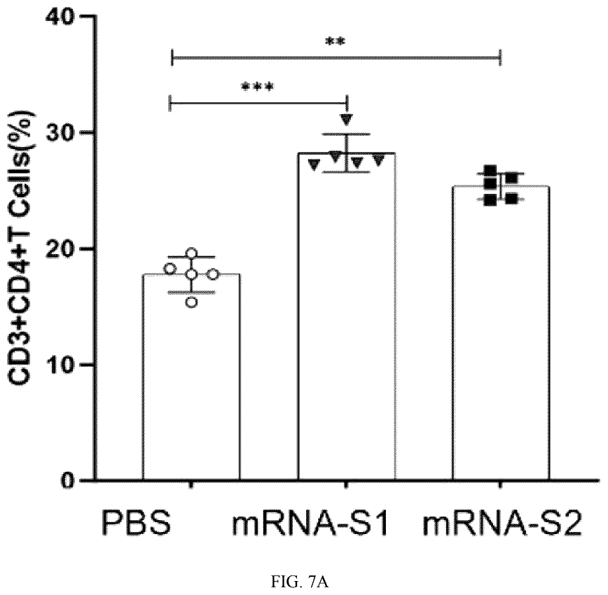

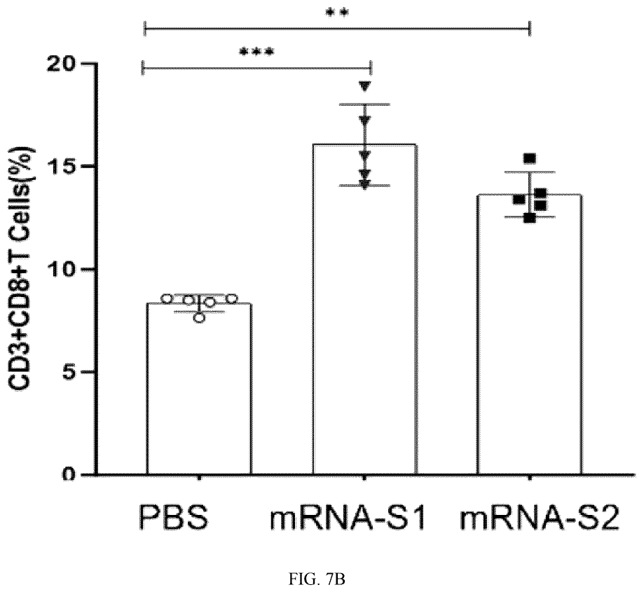

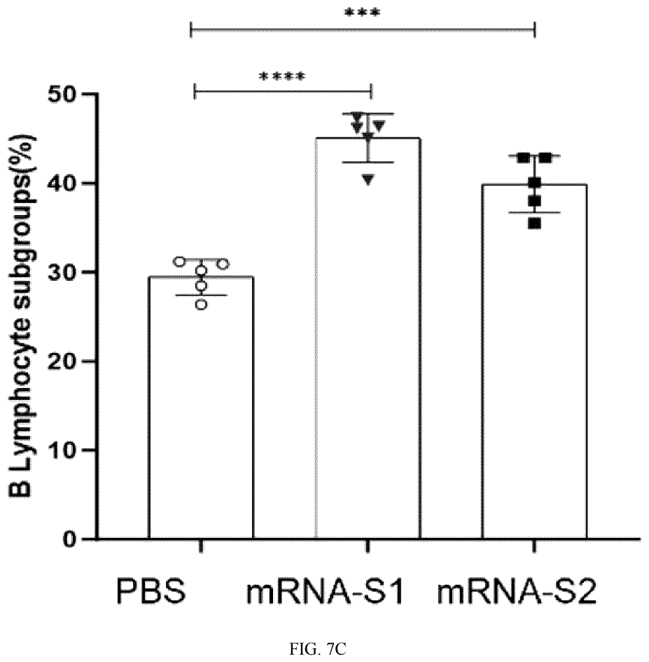

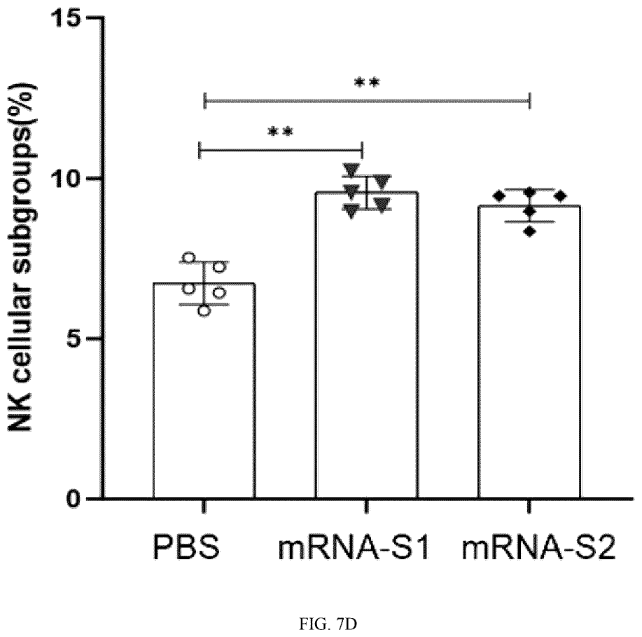

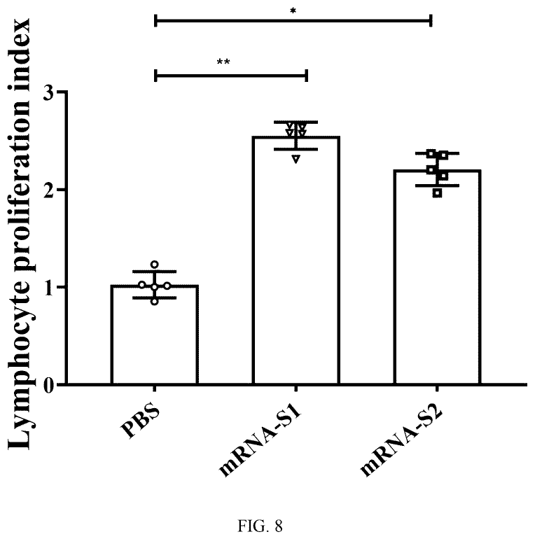

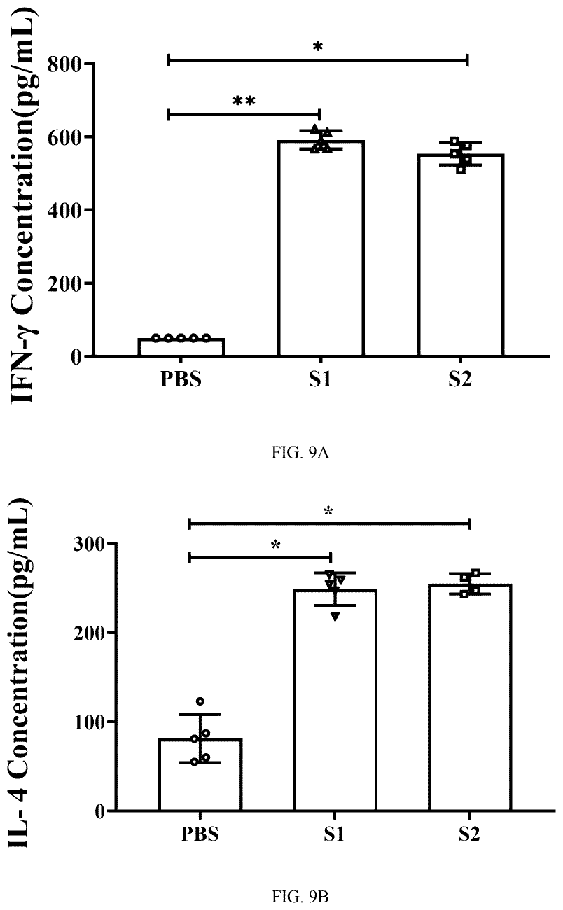

Results are shown in FIG. 3, the sera of mice at 14 days showed a high IgG antibody response, and at 28 days, the immunized mice produced high levels of IgG antibodies. As shown in FIG. 4, the titer of neutralizing antibody is 1:128, and becomes 1:256 after the second immunization. The results indicate that the mRNA-SI and mRNA-S2 vaccines may induce a strong humoral immune response in mice. As shown in FIG. 5-FIG. 6, lymphocytes from mRNA-vaccinated mice produced significantly higher levels of IFN-γ and IL-4 than mice in control groups. To detect mRNA vaccine-induced cellular immune responses, splenic lymphocytes of mice are isolated, and characterization and counts of T-cell, B-cell and NK-cell subsets are analyzed by flow cytometry. The results, as shown in FIG. 7A to FIG. 7D, showed that the percentages of T cell and B cell responses in the mRNA group are higher than those in the PBS control group. As shown in FIG. 8, lymphocyte proliferation in the mRNA group is higher than that in the PBS control group. As shown in FIG. 9A and FIG. 9B, the expression levels of cytokines IFN-γ and IL-4 in the mRNA group are higher than those in the PBS control group. These data indicate that the mRNA vaccine is able to induce humoral and cellular immune responses.

### Embodiment 4 Detection of Antibody Level of Sows Immunized with Lipid Nanoparticle-Encapsulated RNA Vaccine

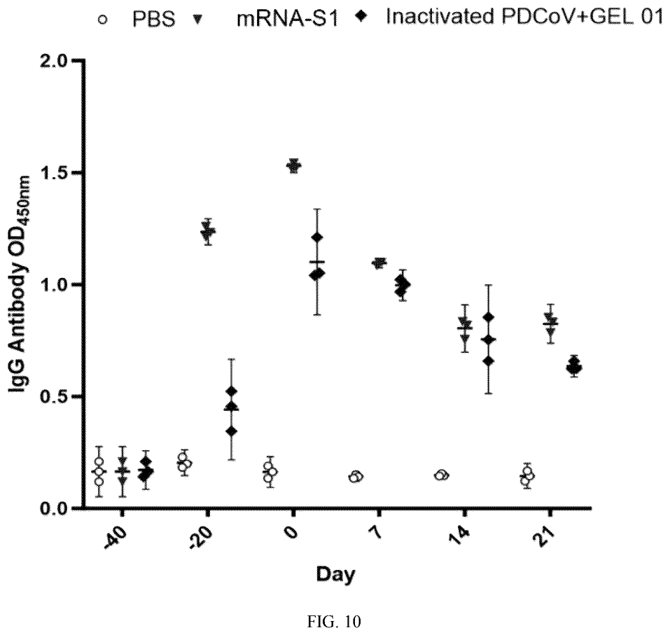

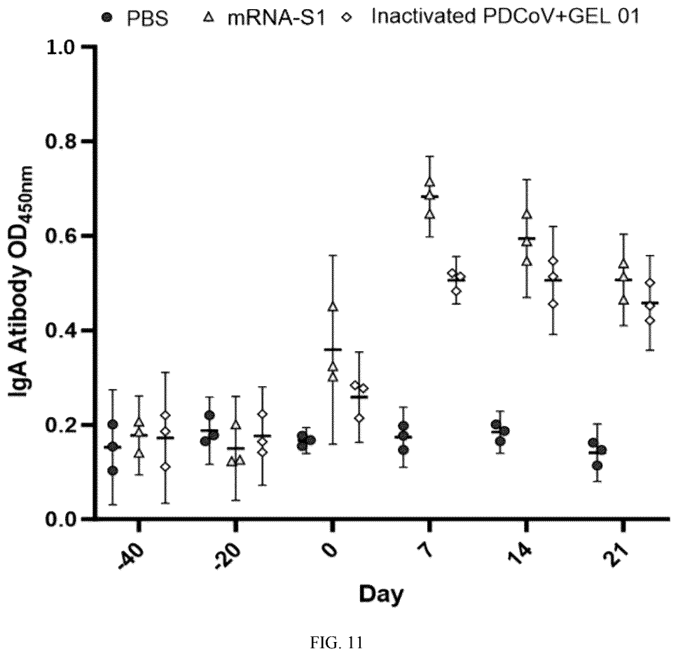

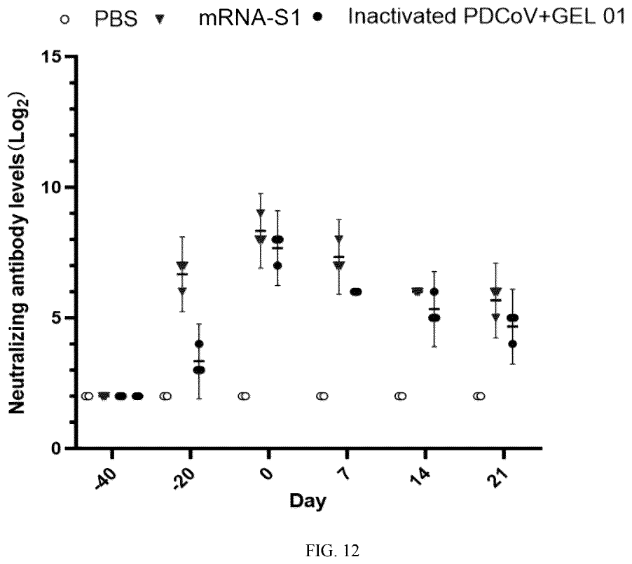

Sows are divided into three groups and immunized with mRNA-S1 vaccine, inactivated PDCoV vaccine and PBS, respectively. Then, sera of sows are collected and anti-PDCoV antibody levels are detected. As shown in FIG. 10-FIG. 11, the results show that the mRNA-SI vaccine induces high titers of IgG and IgA antibodies in sera, and the levels of IgG and IgA antibodies are higher than those in the inactivated vaccine group. The neutralizing ability of serum against PDCoV is determined, and after 14 days, as shown in FIG. 12, the titer of neutralizing antibody in sera is about 1:1024.

Embodiment 5 Detection of the level of passive immunity provided by colostrum of immunized sows to suckling piglets

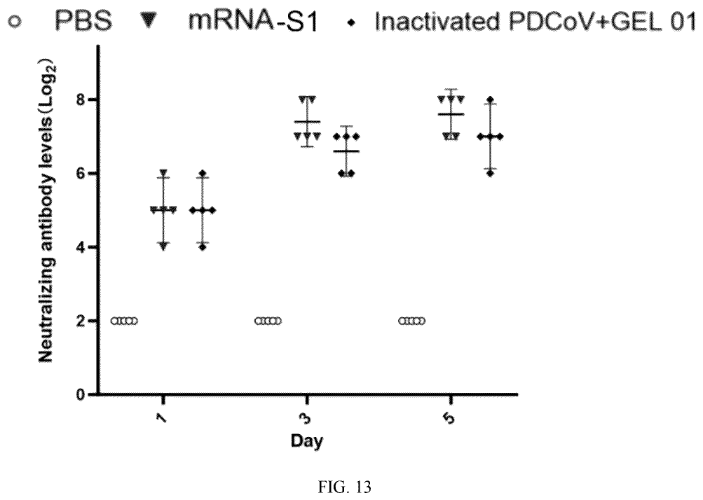

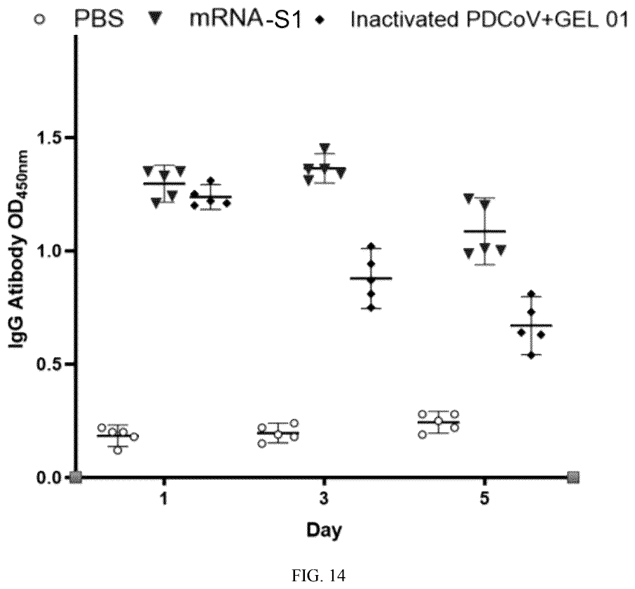

To evaluate the protective effect of the vaccine, the level of passive transfer antibodies in the sera of the piglets is detected. As shown in FIG. 13, the neutralizing antibody level of 5-day-old suckling piglets immunized with the mRNA vaccine is up to 1:256 or more, and the overall neutralizing antibody level is higher than that in the inactivated vaccine group. As shown in FIG. 14, the level of PDCoV-S-binding IgG is high in sera of the piglets, and the mRNA-S1 vaccine induces good mucosal immunity in suckling piglets. Antibodies are found in the sera of piglets born to immunized sows, whereas antibodies are not found in the sera of piglets born to sows in the control group, and it indicates that antibodies are transferred through colostrum. In summary, the mRNA vaccine provides effective passive immunization for newborn piglets and protects them against PDCoV infection.

The above-described embodiments are only descriptions of the preferred manner of the present disclosure, and are not intended to limit the scope of the present disclosure. Without departing from the spirit of the design of the present disclosure, the various deformations and improvements made by the persons of ordinary skill in the field of the technical solutions of the present disclosure shall fall within the scope of protection determined by the claims of the present disclosure.

What is claimed is:

1. An mRNA vaccine encapsulated with lipid nanoparticles for porcine deltacoronavirus, wherein the mRNA vaccine encapsulated with lipid nanoparticles comprises an mRNA vaccine, and lipids for encapsulating the mRNA vaccine, wherein the lipids are formed by mixing ionizable liposome Dlin-MC3-DMA, distearoyl phosphatidylcholine, cholesterol and DMG-PEG2000 liposome;
a molar ratio of the ionizable liposome Dlin-MC3-DMA, the distearoyl phosphatidylcholine, the cholesterol and the DMG-PEG2000 liposome is 55:10:38.5:1.2; and
a nucleotide sequence of the mRNA vaccine is shown in SEQ ID NO: 1.

2. A preparation method of the mRNA vaccine encapsulated with lipid nanoparticles according to claim 1, comprising following steps:
dissolving the ionizable liposome Dlin-MC3-DMA, the distearoyl phosphatidylcholine, the cholesterol and the DMG-PEG2000 liposome in ethanol to prepare a lipid ethanol solution;
diluting the mRNA vaccine with citrate buffer to prepare an mRNA aqueous solution; and
mixing the lipid ethanol solution with the mRNA aqueous solution to prepare the mRNA vaccine encapsulated with the lipid nanoparticles.

3. The preparation method according to claim 2, wherein a volume ratio of the lipid ethanol solution to the mRNA aqueous solution is 1:(2-5).


SEQUENCE LISTING (2 sequences)

SEQ ID NO: 1 - DNA, 3480 bp, synthetic construct
  source 1..3480: mol_type = other DNA
atgcagagagctctattgattatgaccttactttgtctcgttcgagcaaagtttgctgatgatctactcgatttgctcaccttcccgggtgcacatcgcttcttacataaactcacgagtaattccagcagtctctactcgcgggctaataactttgatgttggcgttcttcctggctacgccactaagaacgttaacctcttctcaccacttactaactctactttgccaattaatggccttcatcggagttatcaaccactcatgctgaattgttttactaaaataactaaccacactctcagcatgtatctcctacctagtgatgtacaaacttatagctgcggcggtgccatggttaaataccagacacatgatgcagttcgtatcattttagacctcactgccactgaccacatctctgttgaagtcgttggccagcatggtgaaaattatgtgtttgtttgtagtgagcagtttacctacaccactgcactacacaaatctactttcttctcacttaattctgagctttattgctttactaataacacctacttaggtattcttccacctgatttaactgactttacggtctaccgtactggtcagttctatgctaatggttaccttttaggtactttacctattacggttaactatgttaggttgtatcggggtcaattggctgccaatagtgcccactttgccctagcaaacctaaccgatacacttataacacttaccaataccactatatcgcaaatcacctattgtgataagtcagtagttgattcaatagcatgccagcgctcttctcacgaagtggaggatgggttttactctgaccctaaatctgccgttagagctaggcaacgtactattgttacactacctaagctccctgagcttgaagtagtgcagttaaatatttctgcacacatggattttggcgaagccagacttgacagtgttaccattaatggtaacacatcctattgtgtcactaagccttacttcaggcttgaaactaactttatgtgtacaggttgcactatgaatctgcgcactgatacctgtagttttgacctgtcagcagtaaacaatggcatgtcattctctcaattctgtctaagcactgaatctggtgcttgtgagatgaaaattattgttacctatgtatggaattacttgctaaggcagcgtttgtatgttactgctgtagaaggccagactcacactggaaccacttcagtacatgcaacagacacttctagtgtaatcactgatgtctgcactgactacactatctatggagtctctggtactggcattattaagccatcagatctcttattgcacaatggcatagcattcacctctccaacaggtgagctctatgcatttaaaaatataaccactggcaaaaccctccaggtcttaccgtgtgaaaccccttctcaactgattgtgataaacaacaccgttgtcggtgctatcacatccagtaactcaactgaaaataataggtttactactactattgtcacacctactttcttttattccacaaatgccaccacctttaactgcaccaagcctgttttgtcctatggacccatcagcgtgtgtagtgatggtgcaattgtgggaacatctacattacagaatactcgaccatccatagtttcactatacgatggcgaaattgaaataccatctgcattttccctttctgttcagacagagtatttgcaagttcaagcagagcaagttatagttgattgttctcagtatgtatgcaatggcaacagccgttgtctacaattactggcacaatacacctcagcttgctctaacattgaagcagctctgcattcctctgcacagttggatagcagagagattataaatatgtttcaaacatcaacacagtccttgcagttggctaatattaccaacttcaagggtgactacaattttagcagcataataacccccagaattggtggcagatctgctattgaagaccttctttttaataaagttgttactagtggccttggcactgttgatcaggactacaaagcctgctctagagacatggccatcgctgacttagtttgttcccagtattacaatggcatcatggttctacctggtgttgttgatgctgagaaaatggcaatgtatactggctctcttactggagctatggtatttgggggactgactgctgcagcggcaataccattcgccacggcagtacaagcccgtctcaattatgtcgcactgcaaacaaatgtactacaagaaaaccagaaaattcttgcagaatcatttaaccaagcagttggcaatatatcacttgcactatcttctgttaatgatgccatccagcaaacttctgaggctcttagcaccgtagctattgctattaaaaagattcaaacagttgttaatcagcagggtgaggcattatcacacctgactgcacagctgtcaaacaatttccaagcaatttcgacttctattcaagacatttataaccgtcttgaggaagtagaggctaaccagcaagttgaccgtctcatcacaggacggttggctgcacttaatgcatatgttactcagttactcaatcagatgtctcagattagacaatctcgattgttagctcagcaaaagattaatgagtgtgtcaaatctcagtcgtccagatacggtttctgtggaaatggcacacatatcttctcacttacacagactgcaccaaatggcatatttttcatgcatgcagtacttgtacccaacaaattcacacgtgtcaacgcttctgcgggtatttgtgtggataatacgaaaggctactcattgcagcctcaacttatactctaccagtttaataactcctggagagttacacctagaaatatgtatgaacccagattgccccggcaagctgatttcatacaattaactgattgcagcgttaatttttacaataccaccgctgctaatcttcccaatattatccctgacgttatagatgtcaatcaaacagtcagtgatattattgacaatttacctacagcaacacctcctcagtgggatgttggtatctataacaacactattctcaacctcaccgttgagattaatgatctacaagagcggtctaaaaatctctcacagattgcagatcgtttacaaaattatattgacaatcttaacaatactctagttgaccttgaatggctcaacaaagtggagacttaccttaaatggccgtggtatgtatggcttgccatagccctggctcttattgcatttgtgacaatcctcataacaatctttctctgtactggttgttgtggtggttgttttggttgttgtggcggttgttttggccttttctctaagaagaaaaggtataccgacgaccaaccaacaccgtcctttaagtttaaggaatggtag

SEQ ID NO: 2 - DNA, 3342 bp, synthetic construct
  source 1..3342: mol_type = other DNA
atgaaattcttagtcaacgttgcccttgtttttatggtcgtgtacatttcttacatctatgcggctgatgatctactcgatttgctcaccttcccgggtgcacatcgcttcttacataaactcacgagtaattccagcagtctctactcgcgggctaataactttgatgttggcgttcttcctggctacgccactaagaacgttaacctcttctcaccacttactaactctactttgccaattaatggccttcatcggagttatcaaccactcatgctgaattgttttactaaaataactaaccacactctcagcatgtatctcctacctagtgatgtacaaacttatagctgcggcggtgccatggttaaataccagacacatgatgcagttcgtatcattttagacctcactgccactgaccacatctctgttgaagtcgttggccagcatggtgaaaattatgtgtttgtttgtagtgagcagtttacctacaccactgcactacacaaatctactttcttctcacttaattctgagctttattgctttactaataacacctacttaggtattcttccacctgatttaactgactttacggtctaccgtactggtcagttctatgctaatggttaccttttaggtactttacctattacggttaactatgttaggttgtatcggggtcaattggctgccaatagtgcccactttgccctagcaaacctaaccgatacacttataacacttaccaataccactatatcgcaaatcacctattgtgataagtcagtagttgattcaatagcatgccagcgctcttctcacgaagtggaggatgggttttactctgaccctaaatctgccgttagagctaggcaacgtactattgttacactacctaagctccctgagcttgaagtagtgcagttaaatatttctgcacacatggattttggcgaagccagacttgacagtgttaccattaatggtaacacatcctattgtgtcactaagccttacttcaggcttgaaactaactttatgtgtacaggttgcactatgaatctgcgcactgatacctgtagttttgacctgtcagcagtaaacaatggcatgtcattctctcaattctgtctaagcactgaatctggtgcttgtgagatgaaaattattgttacctatgtatggaattacttgctaaggcagcgtttgtatgttactgctgtagaaggccagactcacactggaaccacttcagtacatgcaacagacacttctagtgtaatcactgatgtctgcactgactacactatctatggagtctctggtactggcattattaagccatcagatctcttattgcacaatggcatagcattcacctctccaacaggtgagctctatgcatttaaaaatataaccactggcaaaaccctccaggtcttaccgtgtgaaaccccttctcaactgattgtgataaacaacaccgttgtcggtgctatcacatccagtaactcaactgaaaataataggtttactactactattgtcacacctactttcttttattccacaaatgccaccacctttaactgcaccaagcctgttttgtcctatggacccatcagcgtgtgtagtgatggtgcaattgtgggaacatctacattacagaatactcgaccatccatagtttcactatacgatggcgaaattgaaataccatctgcattttccctttctgttcagacagagtatttgcaagttcaagcagagcaagttatagttgattgttctcagtatgtatgcaatggcaacagccgttgtctacaattactggcacaatacacctcagcttgctctaacattgaagcagctctgcattcctctgcacagttggatagcagagagattataaatatgtttcaaacatcaacacagtccttgcagttggctaatattaccaacttcaagggtgactacaattttagcagcataataacccccagaattggtggcagatctgctattgaagaccttctttttaataaagttgttactagtggccttggcactgttgatcaggactacaaagcctgctctagagacatggccatcgctgacttagtttgttcccagtattacaatggcatcatggttctacctggtgttgttgatgctgagaaaatggcaatgtatactggctctcttactggagctatggtatttgggggactgactgctgcagcggcaataccattcgccacggcagtacaagcccgtctcaattatgtcgcactgcaaacaaatgtactacaagaaaaccagaaaattcttgcagaatcatttaaccaagcagttggcaatatatcacttgcactatcttctgttaatgatgccatccagcaaacttctgaggctcttagcaccgtagctattgctattaaaaagattcaaacagttgttaatcagcagggtgaggcattatcacacctgactgcacagctgtcaaacaatttccaagcaatttcgacttctattcaagacatttataaccgtcttgaggaagtagaggctaaccagcaagttgaccgtctcatcacaggacggttggctgcacttaatgcatatgttactcagttactcaatcagatgtctcagattagacaatctcgattgttagctcagcaaaagattaatgagtgtgtcaaatctcagtcgtccagatacggtttctgtggaaatggcacacatatcttctcacttacacagactgcaccaaatggcatatttttcatgcatgcagtacttgtacccaacaaattcacacgtgtcaacgcttctgcgggtatttgtgtggataatacgaaaggctactcattgcagcctcaacttatactctaccagtttaataactcctggagagttacacctagaaatatgtatgaacccagattgccccggcaagctgatttcatacaattaactgattgcagcgttaatttttacaataccaccgctgctaatcttcccaatattatccctgacgttatagatgtcaatcaaacagtcagtgatattattgacaatttacctacagcaacacctcctcagtgggatgttggtatctataacaacactattctcaacctcaccgttgagattaatgatctacaagagcggtctaaaaatctctcacagattgcagatcgtttacaaaattatattgacaatcatcatcatcatcatcacagaatgaagcaaattgaagataagattgaagaaattgaatccaagcaaaagaagattgaaaatgaaattgctagaattaagaagtag